(19) United States
(12) Patent Application Publication
(10) Pub. No.: US 2026/0321038 A1
(43) Pub. Date: Oct. 15, 2026

(54) SYSTEM AND METHOD OF IDENTIFYING A TERTIARY LYMPHOID STRUCTURE

(71) Applicant: NUCLEAI LTD., Tel Aviv (IL)

(72) Inventors: Itai MARKOVITS, Ramat Gan (IL); Rivka Becky ARBIV, Tel Aviv (IL)

(21) Appl. No.: 19/704,500

(22) Filed: Jun. 11, 2026

Related U.S. Application Data
(63) Continuation of application No. PCT/IL2024/051170, filed on Dec. 11, 2024.
(60) Provisional application No. 63/608,324, filed on Dec. 11, 2023.

(51) Int. Cl. G06V 20/69; G06T 7/00; G06T 7/11; G06V 10/26; G06V 10/34; G06V 10/764; G06V 10/774; G16H 10/40

(52) U.S. Cl. CPC G06V 20/698; G06T 7/0012; G06T 7/11; G06V 10/267; G06V 10/34; G06V 10/764; G06V 10/774; G06V 20/695; G16H 10/40; G06T 2207/20036; G06T 2207/20081; G06T 2207/30024; G06T 2207/30096

(57) ABSTRACT

A system and method of identifying a Tertiary Lymphoid Structure (TLS) by at least one processor may include receiving an Hematoxylin and Eosin (HE) image, depicting an HE-stained slide comprising a plurality of HE-stained biological cells obtained from a subject; obtaining a candidate region data element, representing a high Lymphocyte Density (LD) region in the HE image; segmenting the high LD region of the candidate region data element, to obtain a plurality of cellular segment data elements, each representing a respective depicted biological cell in the candidate region; for one or more biological cells of the cellular segment data elements, calculating at least one respective cellular structural feature value; and inferring a Machine Learning based LD classification model on the at least one cellular structural feature value of the cellular segment data elements, to classify the candidate region as a TLS region, a TLS sub-class region or a non-TLS region.

### CROSS-REFERENCE TO RELATED APPLICATIONS

This application is a Bypass Continuation of PCT International Application No. PCT/IL2024/051170 having International Filing date of Dec. 11, 2024, titled: “SYSTEM AND METHOD OF IDENTIFYING A TERTIARY LYMPHOID STRUCTURE”, which claims the benefit of U.S. Patent Application No. 63/608,324, filed Dec. 11, 2023, and titled: “SYSTEM AND METHOD OF IDENTIFYING A TERTIARY LYMPHOID STRUCTURE”, both of which are hereby incorporated by reference in their entirety.

### FIELD OF THE INVENTION

The present invention relates generally to the field of image processing and assistive diagnostics'technology. More specifically, the present invention relates to a system and method of identifying a Tertiary Lymphoid Structure (TLS).

### BACKGROUND OF THE INVENTION

A Tertiary Lymphoid Structure (TLS) is a formation of cells, characterized as a high density aggregate of immune cells, typically including B cells, T cells, and Follicular dendritic cells (FDC), in addition to other cell types. While Lymphocyte Density (LD) can be analyzed from Hematoxylin and Eosin (HE, or “H&E”) slides, the presence of the aforementioned cell types requires either multiplex or multiple Immunohistochemistry (IHC) stains.

However, there are several subclasses of TLSs that range from what is referred to herein as Lymphocyte Aggregates (LA) to mature TLS structures with Germinal Centers (GC).

### SUMMARY OF THE INVENTION

While TLS with GC can sometimes be detected from HE slides, it is impossible to distinguish between middle subclasses of TLS, such as “immature TLS” or “non-classical TLS”, as known in the art, using only HE slides.

Classification of TLS, and distinguishing between TLS sub-classes requires understanding of a makeup of immune cells in an LA. In order to do so, currently available systems and methods utilize expensive, and time consuming techniques for staining and analyzing B cells (CD20), T cells (CD3), FDC (CD21), Macrophages (CD68), and High endothelial venules (HEVs) (PNAd).

Embodiments of the invention may facilitate screening of subjects, to classify TLS, and distinguish between TLS sub-classes, based on the commonly used HE staining procedure.

Embodiments of the invention may include a method of identifying a Tertiary Lymphoid Structure (TLS) by at least one processor. Embodiments of the method may include receiving a target Hematoxylin and Eosin (HE) image, depicting an HE-stained slide may include a plurality of HE-stained biological cells obtained from a subject; obtaining a candidate region data element, representing a high Lymphocyte Density (LD) region in the target HE image; segmenting the high LD region of the candidate region data element, to obtain a plurality of cellular segment data elements, each representing a respective depicted biological cell in the candidate region; and for one or more biological cells of the cellular segment data elements, calculating at least one respective cellular structural feature value. According to some embodiments, the at least one processor may subsequently infer a Machine Learning (ML) based LD classification model on the at least one cellular structural feature value of the one or more cellular segment data elements, to classify the candidate region as a TLS region, a TLS sub-class region or a non-TLS region.

Additionally, or alternatively, the at least one processor may segment the high LD region of the candidate region data element, to obtain a plurality of nucleus segment data elements, each representing a nucleus of a respective depicted biological cell in the candidate region. The at least one cellular structural feature value may represent a nucleus structure of one or more nuclei of the nucleus segment data elements.

According to some embodiments, the at least one processor may infer an ML based region detection model on the target HE image, to obtain the candidate region data element. The region detection model may be pretrained to detect regions of high LD in HE images, based on a plurality of training HE images.

According to some embodiments, the at least one processor may train the region detection model by receiving a plurality of training HE images, and a plurality of LD region annotations, corresponding to the plurality of training HE images, wherein at least one LD region annotation defines a region of high LD in a corresponding training HE image; and using the plurality of LD region annotations as supervisory data, to train the region detection model to detect regions of high LD in the plurality of training HE images.

According to some embodiments, the at least one processor may obtain or calculate the candidate region data element by obtaining a plurality of cell type labels, where each cell type label defines a cell type of a respective biological cell, depicted in the target HE image. The at least one processor may produce a density map, representing density of lymphocyte cells based on the plurality of cell type labels, and identify high LD regions in the target HE image based on the density map.

According to some embodiments, the at least one processor may obtain a cell type label by segmenting the target image to one or more cell segments, where each cell segment may include a cell depicted in the target image. The at least one processor may then infer an ML based cell classification model on the one or more cell segments to obtain the cell type labels. The cell classification model may be pretrained to classify cell segments in HE images, based on a plurality of annotated, training cell segments.

According to some embodiments, the at least one processor may train the cell classification model by receiving a plurality of training cell segments, each depicting a biological cell; receiving a plurality of cell type annotations, each defining a cell type of a respective biological cell, depicted in the training HE image; and using the cell type annotations as supervisory data for training the cell classification model to classify biological cells depicted in the training cell segments, according to cell types.

Additionally, or alternatively, the at least one processor may infer the region detection model on a training HE image of the plurality of training HE images, to obtain a candidate region data element, representing a high LD region in the training HE image; obtain a plurality of cellular segment data elements, each representing a respective biological cell in the high LD region of the candidate region data element; for one or more cells of the cellular segment data elements, calculate at least one respective cellular structural feature value; receive a TLS annotation data element, labelling the high LD region of the candidate region data element as representing either (i) a TLS region, (ii) a TLS sub-class region, or (iii) a non-TLS region; and use the TLS annotation data element as supervisory data for training the LD classification model to classify the high LD region of the candidate region data element, based on the cellular structural feature values. The high LD region may be classified, for example, as a TLS region, a TLS sub-class region, a non-TLS region, and the like.

According to some embodiments, the cellular structure features may include, for example a cell's size, a cell's roundness, a cell's eccentricity, a cell's convexity, a ratio between different areas of the cell, a cell's mean intensity, a cell's intensity variance, a cell's intensity range, a cell's color, a cell's color range, and the like.

Additionally, or alternatively, the cellular structure features may represent structural features of cell-specific nuclei. Such structural features may include, for example nucleus convexity, nucleus eccentricity, nuclear area, nuclear solidity, nuclear color intensity variance, nuclear color intensity range, nuclear color intensity mean value, and the like.

Additionally, or alternatively, the cellular structure feature may be a summary cellular structure feature, such as a mean, a median and a standard deviation of a plurality of cell-specific cellular structure features, pertaining to a plurality of cells in one or more candidate regions.

According to some embodiments, the at least one processor may calculate at least one morphology data element, representing a morphology of the candidate region; and infer the ML based LD classification model further on the at least one morphology data element, to classify the candidate region e.g., as a TLS region, a TLS sub-class region, or a non-TLS region.

According to some embodiments, the morphology data element may include, for example an area of the candidate region, a cell count within the candidate region, a convexity of a polygon representing a circumference of the candidate region, a solidity of the polygon, and an eccentricity of the polygon.

According to some embodiments, the at least one processor may produce a status notification, based on the classification of one or more candidate regions in one or more slides. The status notification may include, for example a diagnosis of the subject, a prognosis of the subject, a suggested treatment for the subject, and the like.

Additionally, or alternatively, the at least one processor may perform selective computation of cellular structural features. For example, the at least one processor may, for at least one cellular segment data element, classify the respective, depicted biological cell according to a cell type of a predefined set of cell types; and selectively calculate the cellular structural feature value based on the classification.

Embodiments of the invention may include a system for identifying a Tertiary Lymphoid Structure (TLS). Embodiments of the system may include a non-transitory memory device, wherein modules of instruction code may be stored, and at least one processor associated with the memory device, and configured to execute the modules of instruction code. Upon execution of said modules of instruction code, the at least one processor may be configured to: receive a target Hematoxylin and Eosin (HE) image, depicting an HE-stained slide may include a plurality of HE-stained biological cells; obtain a candidate region data element, representing a high Lymphocyte Density (LD) region in the target HE image; segment the candidate region, to obtain a plurality of nucleus segment data elements, each representing a nucleus of a respective depicted biological cell in the candidate region; for one or more nuclei of the nucleus segment data elements, calculate at least one respective nuclear structural feature value; and infer a Machine Learning (ML) based LD classification model on the at least one nuclear structural feature value of the one or more nucleus segments, to classify the candidate region as a TLS region, a TLS sub-class region, or a non-TLS region.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a block diagram, depicting a computing device which may be included in a system for identifying a TLS in an HE slide, according to some embodiments;

FIG. 2 is an image depicting a portion of an HE slide, including a region identified as a TLS region by embodiments of the invention;

FIG. 3 is a schematic diagram depicting a typical formation of immune cells of different types within a TLS structure;

FIG. 4 is a schematic block diagram depicting a system for identifying a TLS in an HE slide, according to some embodiments;

FIG. 5 is a schematic block diagram depicting a candidate region identifier module, which may be included in a system for identifying a TLS in an HE slide, according to some embodiment;

FIG. 6 is a schematic flow diagram depicting a method of identifying a TLS in an HE slide, according to some embodiments;

FIG. 7 is a schematic flow diagram depicting a method of training an LD classifier to classify a candidate Region of Interest (ROI) as a TLS region, a TLS sub-class region or a non-TLS region in an HE slide, according to some embodiments;

FIG. 8A is an image of an HE slide, annotated to include a color-coded cell types, according to some embodiments;

FIGS. 8B and 8C respectively depict an overview image and a zoomed-in image of the HE slide of FIG. 8A, where nuclei of biological cells are segmented according to some embodiments;

FIG. 9A depicts three examples of HE slides that may be analyzed by embodiments of the invention to detect TLS regions;

FIG. 9B depicts three images, corresponding to those of FIG. 9A, where candidate regions of interest were identified by embodiments of the invention;

FIG. 9C depicts three lymphocyte density maps, produced by embodiments of the invention, in respect to the corresponding images of FIG. 9A; and

FIG. 10 is a schematic flow diagram depicting a method of identifying a TLS region in an HE slide obtained from a subject, by at least one processor, according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term “set” when used herein may include one or more items.

Unless explicitly stated, the method embodiments described herein are not constrained to a particular order or sequence. Additionally, some of the described method embodiments or elements thereof can occur or be performed simultaneously, at the same point in time, or concurrently.

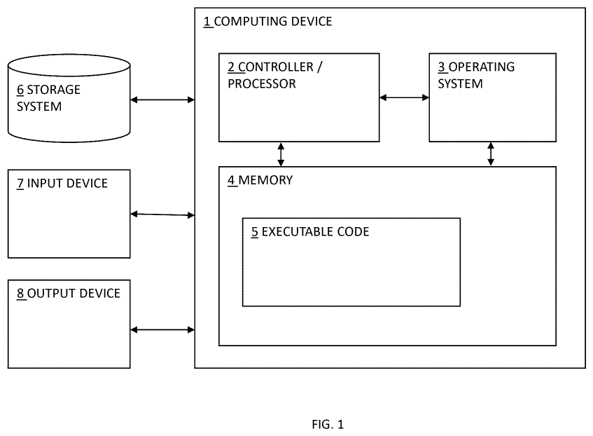

Reference is now made to FIG. 1, which is a block diagram depicting a computing device, which may be included within an embodiment of a system for identifying a TLS in an HE slide, according to some embodiments.

Computing device 1 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 1 may be included in, and one or more computing devices 1 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 1, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random-Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task, or script. Executable code 5 may be executed by processor or controller 2 possibly under control of operating system 3. For example, executable code 5 may be an application that may identify a TLS in an HE slide of a subject as further described herein. Although, for the sake of clarity, a single item of executable code 5 is shown in FIG. 1, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Data pertaining to one or more slides obtained from one or more subjects may be stored in storage system 6 and may be loaded from storage system 6 into memory 4 where it may be processed by processor or controller 2. In some embodiments, some of the components shown in FIG. 1 may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components, or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 1 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 1 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

The term neural network (NN) or artificial neural network (ANN), e.g., a neural network implementing a machine learning (ML) or artificial intelligence (AI) function, may be used herein to refer to an information processing paradigm that may include nodes, referred to as neurons, organized into layers, with links between the neurons. The links may transfer signals between neurons and may be associated with weights. A NN may be configured or trained for a specific task, e.g., pattern recognition or classification. Training a NN for the specific task may involve adjusting these weights based on examples. Each neuron of an intermediate or last layer may receive an input signal, e.g., a weighted sum of output signals from other neurons, and may process the input signal using a linear or nonlinear function (e.g., an activation function). The results of the input and intermediate layers may be transferred to other neurons and the results of the output layer may be provided as the output of the NN. Typically, the neurons and links within a NN are represented by mathematical constructs, such as activation functions and matrices of data elements and weights. At least one processor (e.g., processor 2 of FIG. 1) such as one or more CPUs or graphics processing units (GPUs), or a dedicated hardware device may perform the relevant calculations.

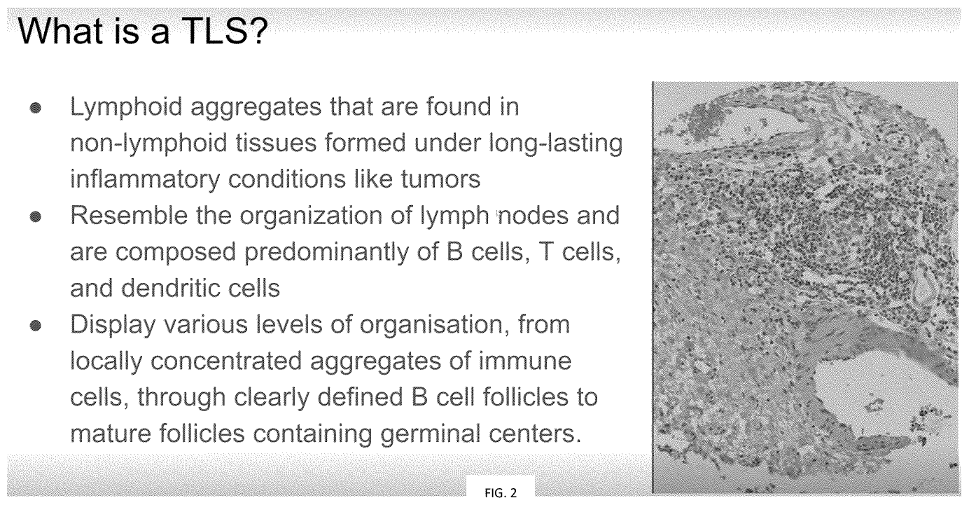

Reference is now made to FIG. 2 which is an image depicting a portion of an HE slide obtained from a subject. FIG. 2 also includes a region, bordered by a yellow line, identified by embodiments of the invention as a TLS region.

A trained expert, e.g., a pathologist may sometimes be able to identify the marked region as a high-density lymphoid aggregate, that was found in a non-lymphoid tissue. The expert may thereby suspect that the high-density lymphoid aggregate is a TLS region, indicating presence of a tumor. However the expert may not be able to confirm this suspicion, nor determine characteristics of the suspected tumor without additional information regarding the types and locations of specific cells within the suspected region.

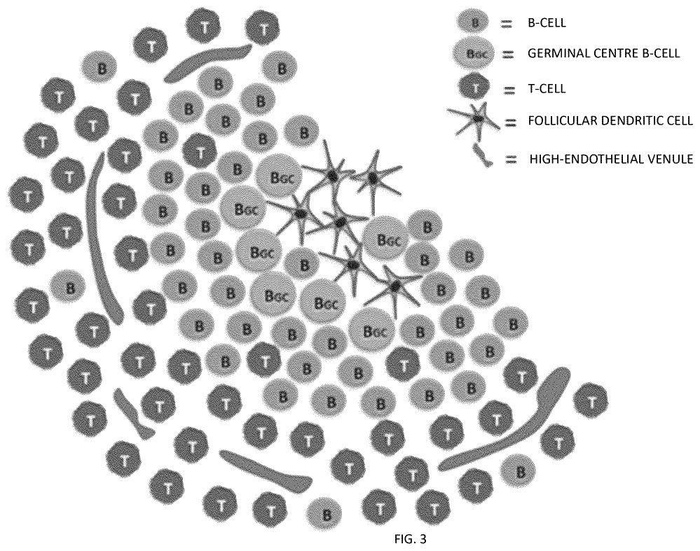

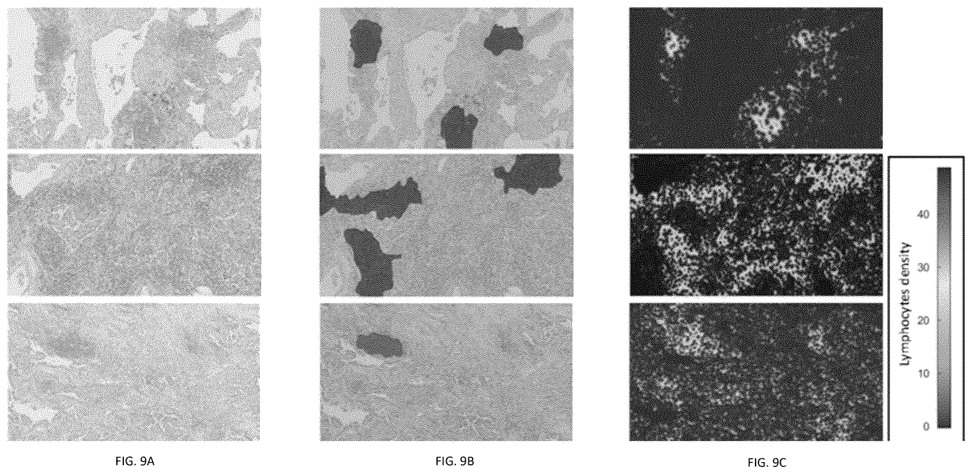

Reference is now made to FIG. 3 which is a schematic diagram depicting a typical formation of immune cells of different types within a TLS structure. As shown in this schematic example, a formation of a TLS region is typically characterized by a layered, concentric structure of immune cells, where an aggregation of FDC cells are typically encapsulated by subsequent layers of Germinal center B cells, followed by T cells, and high Endothelial venules. A trained expert, e.g., a pathologist may require an Immunofluorescence (IF) staining (also referred to herein as Multiplex staining), or IHC staining to learn the identification of depicted cell types, and thereby classify the observed structure e.g., as TLS, or non-TLS Lymphocyte Aggregate. However, as known in the art, Multiplex staining and IHC staining techniques are not readily available, and are expensive and complicated to implement. Therefore, Multiplex staining and IHC staining techniques are not suitable for diagnostic screening of a large number of subjects. Examples for Multiplex staining and IHC staining are provided herein, e.g., in relation to FIG. 9.

Therefore, and as elaborated herein, embodiments of the invention may include a method of automated analysis of commonly available HE slides, to determine a candidate region (e.g., one that is suspected to include TLS), classify that region (e.g., as TLS, or non-TLS LA), and/or diagnose or screen subjects based on this analysis.

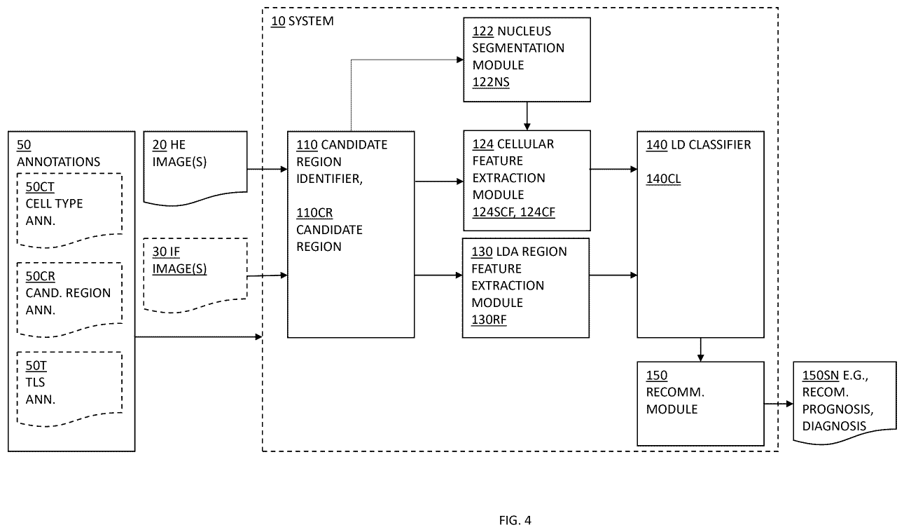

Reference is now made to FIG. 4, which is a schematic block diagram depicting a system 10 for identifying a TLS in an HE slide, according to some embodiments.

According to some embodiments of the invention, system 10 may be implemented as a software module, a hardware module, or any combination thereof. For example, system may be or may include a computing device such as element 1 of FIG. 1, and may be adapted to execute one or more modules of executable code (e.g., element 5 of FIG. 1) to identify a TLS in an HE slide of a subject, as further described herein. As shown in FIG. 4, arrows may represent flow of one or more data elements to and from system 10 and/or among modules or elements of system 10. Some arrows have been omitted in FIG. 4 for the purpose of clarity.

As shown in FIG. 4, system 10 may receive a target HE image 20 depicting an HE-stained slide. The HE-stained slide may include a plurality of HE-stained biological cells obtained from a subject, such as a human patient, or a veterinary patient. The terms HE slide 20 and HE image 20 may be used herein interchangeably. The term “target” may be used in this context to indicate an HE slide 20 of interest, upon which a preconfigured system 10 may be applied to, or inferred upon, to identify a TLS therein.

According to some embodiments, system 10 may include a candidate region identifier module 110, configured to obtain a candidate region data element 110CR, representing a high Lymphocyte Density (LD) region in the target HE image. The terms candidate region 110CR and high LD region 110CR may be used herein interchangeably, to indicate a region of interest to be analyzed by embodiments of the invention for presence of TLS.

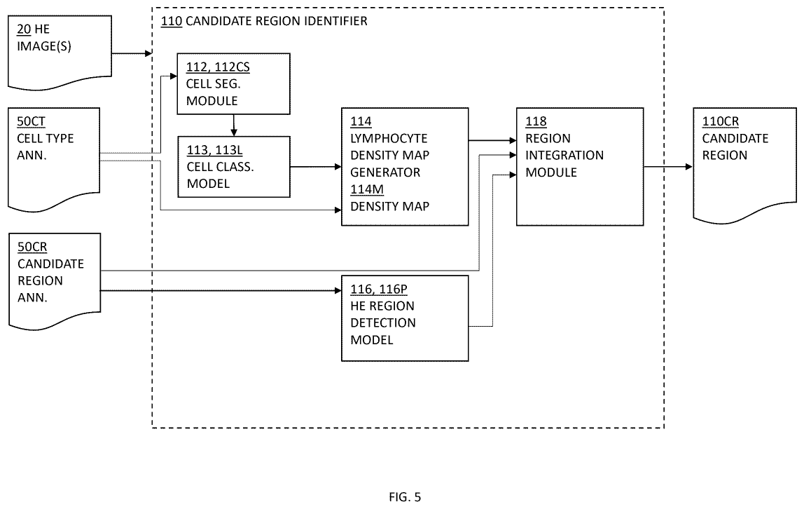

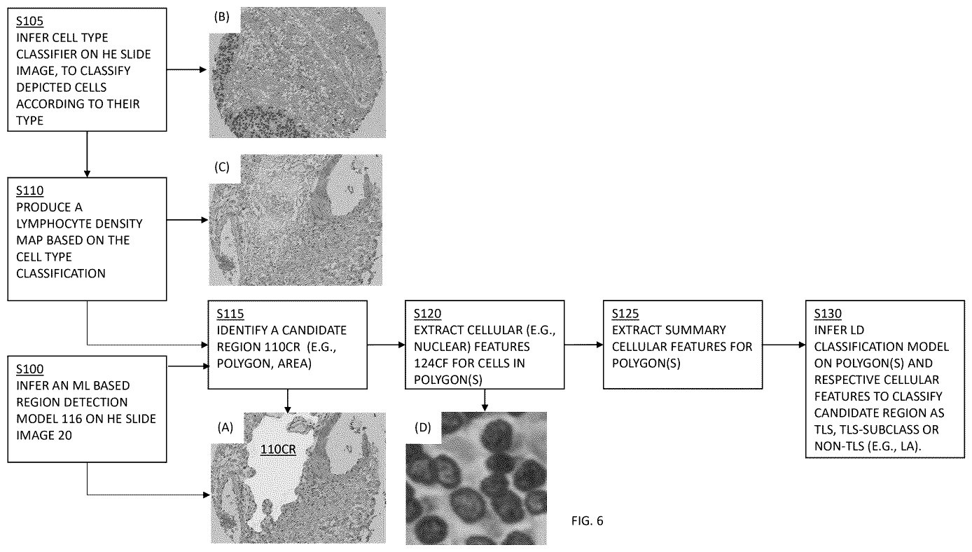

Reference is also made to FIG. 5, which is a schematic block diagram depicting a candidate region identifier module 110 (or “candidate module 110” for short) that may be included in a system 10 for identifying a TLS in an HE slide 20. Reference is also made to FIG. 6, which is a schematic flow diagram depicting a method of identifying a TLS in HE slide 20, according to some embodiments of the invention.

As shown in FIG. 5, candidate module 110 may act in one or more paths to obtain, or identify candidate regions 110CR in HE image 20.

For example, system 10 may be associated with, or included in a computing device (e.g., computing device 1 of FIG. 1) pertaining to an expert user (e.g., a pathologist). Candidate module 110 may be configured to present HE slide 20 to the user, e.g., via a User Interface (UI) of system 10 (e.g., input device 7 and/or output device 8 of FIG. 1, such as a touch-screen). Candidate module 110 may prompt the user via the UI 7/8 to mark or annotate a candidate region of interest in HE image 20, and may subsequently receive a candidate region annotation 50CR via the UI 7/8 from the user. Candidate region annotation 50CR (also referred to as LD region annotations 50CR) may include, for example a marking, or definition (e.g., an area, a circumference, etc.) of a candidate LD region of interest in HE slide 20.

Additionally, or alternatively, candidate module 110 may include an ML based region detection model 116, that may be pretrained to detect candidate regions of high LD 110CR in HE images 20, based on a plurality of training HE images 20.

In other words, during a training stage, system 10 may receive (e.g., via input device 7) a plurality of training HE images 20. Additionally, system 10 may receive (e.g., from an expert user) a plurality of LD region annotations 50CR corresponding to the plurality of training HE images 20. On or more (e.g., each) LD region annotation 50CR may define, or represent a region (e.g., an area, or circumference) of high LD in a corresponding training HE image 20. System 10 may train region detection model 116, using the plurality of LD region annotations 50CR as supervisory data, to train region detection model 116, to detect or predict 116P regions of high LD, e.g., candidate regions 110CR, in the plurality of training HE images 20.

As shown in step S100 FIG. 6, during an inference stage, candidate module 110 may infer, or apply pretrained region detection model 116 on target HE image 20, to predict 116P, or obtain a candidate region data element 110CR. An example of candidate region data element 110CR is shown by a color-filled area in panel (A) of FIG. 6.

It may be appreciated that the training stage of detection model 116 may precede the inference stage, where detection model 116 may be applied to identify, or predict 116P regions of high LD 110CR in target HE images 20. Additionally, or alternatively, system 10 may train detection model 116 continuously, e.g., repeatedly over time as HE images 20 and/or annotations 50CR are received. In such embodiments, the training and inference stages of detection model 116 may be performed intermittently.

Additionally, or alternatively, candidate module 110 may employ another path to calculate density of lymphocyte cells depicted in target HE image 20, and thereby obtain, or identify candidate regions 110CR in HE image 20.

For example, candidate module 110 may include a cell segmentation module 112, and an ML based cell type classification model 113.

Cell segmentation module 112 may be implemented as an ML-based segmentation model, such as a NN model. The ML-based segmentation model may be configured, or pretrained to segment target image 20 to one or more (e.g., a plurality) of cell segments 112CS, each including, or depicting a cell that was depicted in target image 20. Cell segmentation module 112 may be trained based on an appropriate training dataset, as known in the art: The training dataset may include labels, annotations, or markings of cells in a plurality of images, to allow the ML-based segmentation model to segment incident, previously unseen target HE images 20 into cell segments 112CS.

Candidate module 110 may subsequently infer ML based cell type classification model 113 on the one or more cell segments 112CS, to obtain cell type labels 113L, where each cell type label may define or represent a cell type of a respective biological cell, depicted in target HE image 20. Pertaining to the example of FIG. 3, labels 113L may each represent a type or class of a depicted cell such as a B-cell, a T-cell, a Germinal center B-cell, an FDC cell, a high endothelial venule, and the like.

According to some embodiments, cell type classification model 113 may be pretrained to classify cell segments 112CS in HE images 20, based on a plurality of annotated, training cell segments 112CS.

In other words, during a training stage, cell type classification model 113 may receive (e.g., via segmentation module 112) a plurality of training cell segments 112CS, each depicting a biological cell. Cell type classification model 113 may also receive, e.g., from an expert user, via input device 7, a plurality of cell type annotations 50CT. Each cell type annotation 50CT may define a cell type of a respective biological cell, depicted in the training HE image (e.g., B-cells, T-cells, etc.). Candidate module 110 may use the cell type annotations 50CT as supervisory data for training the cell type classification model 113 to classify, or label 113L biological cells depicted in the training cell segments 112CS, according to their cell types.

As shown in step S105 of FIG. 6, during an inference stage, candidate region 110 may employ cell segmentation module 112, and infer cell type classification module 113 no HE slide 20 to classify cells depicted in target HE image 20 according to their type. In other words, candidate region 110 may obtain a plurality of cell type labels 113L, each identifying a type of a cell depicted in HE image 20. A color-coded result of cell type classification is shown in panel (B) of FIG. 6.

As shown in FIG. 5, candidate module 110 may include a lymphocyte density map generator 114, configured to produce a density map 114M, based on the plurality of cell type classification labels 113L. An example of such a map is provided herein (e.g., in relation to FIG. 9C)

In some embodiments, and as shown in step S110 of FIG. 6, density map generator 114 may produce a density map 114M that may be, or may include a matrix data element, where each entry may represent density (e.g., a number of depicted cells within a region depicted in HE image 20), as shown in the example of panel (C) of FIG. 6.

Additionally, or alternatively, density map 114M may include a plurality of matrix data elements, each representing density of a specific type of lymphocyte cells (e.g., B-cells, T-cells, etc.) depicted in target HE image 20.

As shown in FIG. 5, candidate module 110 may include a region integration module 118, adapted to integrate between the different paths of candidate region identifications as elaborated herein, to identify at least one candidate region 110CR.

For example, and as shown in step S115 of FIG. 6, region integration module 118 may identify a high LD candidate region 11CR in target HE image 20 as a function (e.g., a weighted sum) of (i) annotation 50CR provided from a user via input device 7, (ii) predicted regions 116P of high LD from region detection model 116, and/or (iii) density map 114M. Region integration module 118 may represent candidate region 110CR as an area (e.g., a filled area) in HE image 20. Additionally, or alternatively, Region integration module 118 may represent candidate region 110CR as a polygon at a circumference of candidate region 110CR in HE image 20. In this context, the words circumference and polygon may be used interchangeably.

According to some embodiments, system 10 may include a nucleus segmentation module 122, configured to segment the high LD region of the candidate region data element 110CR, so as to obtain a plurality of nucleus segment data elements 122NS. Each nucleus segment data element 122NS may represent a nucleus of a respective depicted biological cell in the candidate region 110CR of HE image 20.

Nucleus segmentation module 122 may be implemented as an ML-based segmentation model, such as a NN model. The ML-based segmentation model may be pretrained to segment, or identify cell nuclei by a supervised training scheme, based on an appropriate training dataset, as known in the art. The training dataset may include labels, annotations, or markings of nuclei in a plurality of images, to allow the ML-based segmentation model 122 to segment nuclei in incident, previously unseen HE images 20. An example of segment data element 122NS, representing polygons of segmented nuclei of depicted biological cells is provided in panel (D) of FIG. 6.

As shown in FIG. 4, system 10 may include a cellular feature extraction module 124. Module 124 may be configured to calculate, for one or more cell segments 112CS at least one respective cellular structural feature value 124CF. Additionally, or alternatively, module 124 may be configured to calculate, for one or more nuclei of the nucleus segment data elements 122NS, at least one respective cellular structural feature value 124CF that represents a structure of the depicted nucleus.

For example, a cellular structural feature 124CF value may include, or represent a feature of structure of a specific cell. Such cellular features 124CF may include, for example a value of the cell's size, a value of the cell's roundness or eccentricity, a value of the cell's convexity, a ratio between different depicted areas of the cell, a value of the cell's intensity or intensity variance, a value of the cell's intensity range, a value of the cell's color, or color range, a value of the cell's intensity mean value, and the like.

In another example, a cellular structural feature 124CF value may include, or represent a nuclear structure of a specific cell. Such a cellular features 124CF may include, for example a value of nucleus convexity, a value of nucleus eccentricity, a value of nuclear area, a value of nuclear solidity, a value of nuclear color intensity variance, a value of nuclear color intensity range, and a nuclear color intensity mean value, and the like.

The extraction of cellular (e.g., nuclear) structural feature 124CF is also shown in step S120 and adjoint panel (D) of FIG. 6, where nuclei of cells in HE image 20 are segmented 122NS, e.g., as presented by colored circumference polygons, and cell-specific cellular structure feature 124CF values, such nucleus convexity and nucleus area are calculated based on these polygons.

Additionally, or alternatively, the cellular structure feature 124CF may be a summary cellular structure feature, representing a statistical value of morphology of a plurality of cells or nuclei within one or more candidate regions 110CR. For example, summary cellular structure feature 124SCF values may include a mean value of cell-specific cellular structure feature 124CF values (e.g., nucleus convexity), pertaining to a plurality of cells in one or more candidate regions 110CR. In a similar manner, summary cellular structure feature 124SCF values may include a median value, and/or a standard deviation value of cell-specific cellular structure feature values 124CF, pertaining to a plurality of cells in one or more candidate regions 110CR.

According to some embodiments, cellular feature extraction module 124 may calculate cellular structural feature value 124CF selectively, for specific cell segments 112CS, to improve efficiency of the structural feature value 124CF computation process.

For example, for at least one cellular segment data element, cellular feature extraction module 124 may perform initial classification of a respective depicted biological cell according to a cell type of a predefined set of cell types (e.g., cell types of interest). cellular feature extraction module 124 may then selectively calculate the cellular structural feature value based on this classification. For example, cellular feature extraction module 124 may filter out depicted cells of specific location or size, to obtain a subset of cellular segments 112CS that depict cells of interest, and only extract structural feature values 124CF of the subset of cellular segments 112CS.

Additionally, or alternatively, and as shown in FIG. 4, system 10 may further include an LDA region feature extraction module 130. Module 130 may be configured to calculate at least one morphology data element 130RF, representing a morphology of at least one specific candidate region or area 110CR.

For example, morphology data element 130RF may include a value of an area of the candidate region 110CR, a cell count within the candidate region 110CR, a convexity of a polygon representing a circumference of the candidate region 110CR, a solidity of such polygon of candidate region 110CR, and an eccentricity of such polygon of candidate region 110CR.

As shown in FIG. 4, system 140 may include a machine-learning (ML) based LD classification model 140, also referred to herein as a TLS classifier model 140.

As elaborated herein, LD classification model 140 may be pretrained, or configured to classify a candidate region 110CR to at least one category or classification 140CL, based on one or more cellular structural feature values 124CF and/or one or more morphology data elements 130RF.

According to some embodiments, system 10 may (e.g., during an inference stage) infer LD classification model 140 on the at least one cellular structural feature value 124CF of the one or more nucleus segment 122NS or cell segments 112CS data elements, to classify the candidate region 110CR to one of a plurality of classifications or categories 140CL.

Additionally, or alternatively, system 10 may (e.g., during the inference stage) further infer ML based LD classification model 140 on at least one morphology feature data element 130RF, to classify the candidate region 110CR to one of a plurality of classifications or categories 140CL.

For example, classification model 140 may classify or assign candidate region 110CR to a category 140CL of a TLS region, or a category 140CL of a non-TLS region.

Additionally, or alternatively, classification model 140 may classify or assign candidate region 110CR to a category or classification 140CL of a TLS sub-class region, such as a mature TLS structures having germinal centers (GC), a category or classification 140CL of immature TLS structures, a category or classification 140CL of stromal inflammation, and the like.

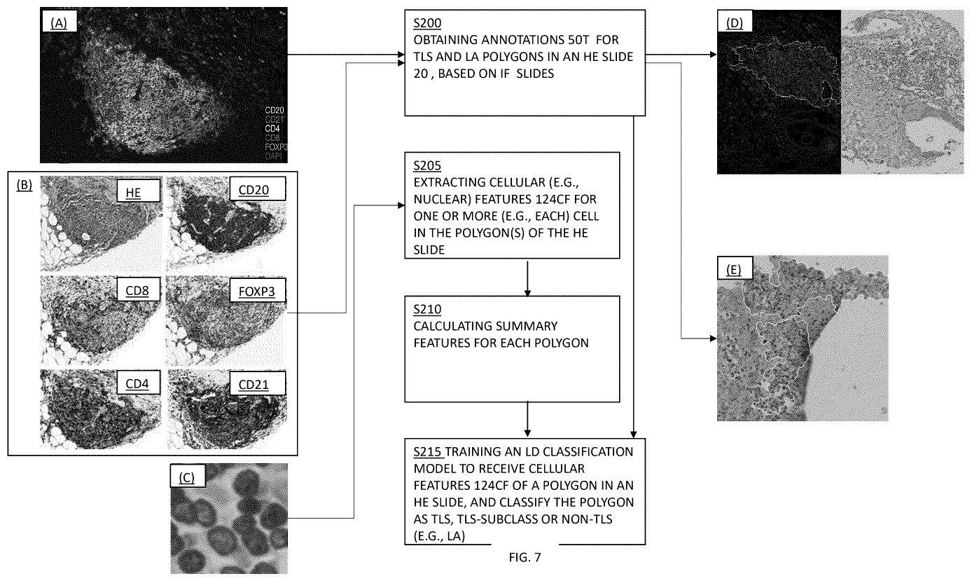

Reference is now made to FIG. 7 which is a schematic flow diagram depicting a method of training an LD classifier to classify, or assign classification 140CL to a candidate Region of Interest (ROI) 110CR in an HE slide, according to some embodiments. Classification 140CL may, for example include a TLS region, a TLS sub-class region and/or a non-TLS region.

According to some embodiments, system 10 may (e.g., as part of a training stage of LD classifier 140) infer region detection model 116 on a training HE image of the plurality of training HE images, to obtain a candidate region data element 110CR, representing a high LD region in the training HE image 20. Additionally, or alternatively, system 10 may (e.g., as part of the training stage) employ lymphocyte density map generator 114 as elaborated herein to obtain a density map 114M, showing regions 110CR of high lymphocyte density in the training HE image 20.

System 10 may proceed to employ nucleus segmentation module 122 as elaborated herein, to obtain a plurality of nucleus segment data elements 122NS, each representing a nucleus of a respective biological cell in the high LD region of the candidate region data element 110CR.

System 10 may subsequently employ, for one or more nuclei of the nucleus segment data elements 122NS feature extraction module 124, to calculating at least one respective cellular (e.g., nuclear) structural feature value 124CF, as elaborated herein. In other words, and as shown in step S205 of FIG. 7, system 10 may extract cellular features values 124CF for one or more (e.g., each) cell (or nucleus) in the candidate region 110CR polygon(s) of HE slide 20.

Additionally, or alternatively, and as shown in step S210 of FIG. 7, system 10 may calculate one or more summary cellular features values 124SCF. For example, summary cellular features values 124SCF may represent a statistical value of cell-specific cellular or nuclear structure feature 124CF values in a candidate region 110CR, as elaborated herein.

Additionally, or alternatively, system 10 may employ LD region feature extraction module 130 to calculating at least one respective morphological feature value 130RF of candidate region 110CR, as elaborated herein.

According to some embodiments, system 10 may receive (e.g., via input device 7, as part of a training stage of LD classifier 140) a TLS annotation data element 50T, labelling the high LD region of candidate region data element 110CR as representing either (i) a TLS region, (ii) a TLS sub-class region, or (iii) a non-TLS region classifications 140CL.

As shown in step S200 of FIG. 7, system 10 may obtain (e.g., from an expert user) annotations 50T for TLS and LA polygons of candidate region 110CR in an HE slide 20, based on one or more Immunofluorescence slides.

For example, HE image 20 may be presented, via output device 8 to an expert pathologist, juxtaposed an IHC staining (panel (B) of FIG. 7) or an Immunofluorescence (panel (A) of FIG. 7) or Multiplex staining (as commonly referred to in the art) of the same slide. System 10 may prompt the expert to label or annotate portions of HE image 20 based on their experience according to the appropriate classification 140CL. Example for such annotation is shown in panels D and E of FIG. 7. System 10 may subsequently receive TLS annotation data element 50T from the expert pathologist in response to this prompt.

System 10 may subsequently use TLS annotation data element 50T as supervisory data for training LD classification model 140, to classify the high LD region of the candidate region data element 110CR as a TLS region, a TLS sub-class region, or a non-TLS region, based on the cellular structural, feature values 124CF and/or morphological features 130RF.

In other words, and as shown in step S215 of FIG. 7, system 10 may train LD classification model 140 based on TLS annotation data elements 50T, to receive cellular features 124CF, summary cellular features 124SCF, and/or morphological features 130RF of a polygon defining a candidate region 110CR in HE slide 20, and to classify 140CL the polygon of candidate region 110CR based on the received features. Classify 140CL may, for example define candidate region 110CR as a TLS region, a TLS-subclass region or a non-TLS (e.g., LA) region.

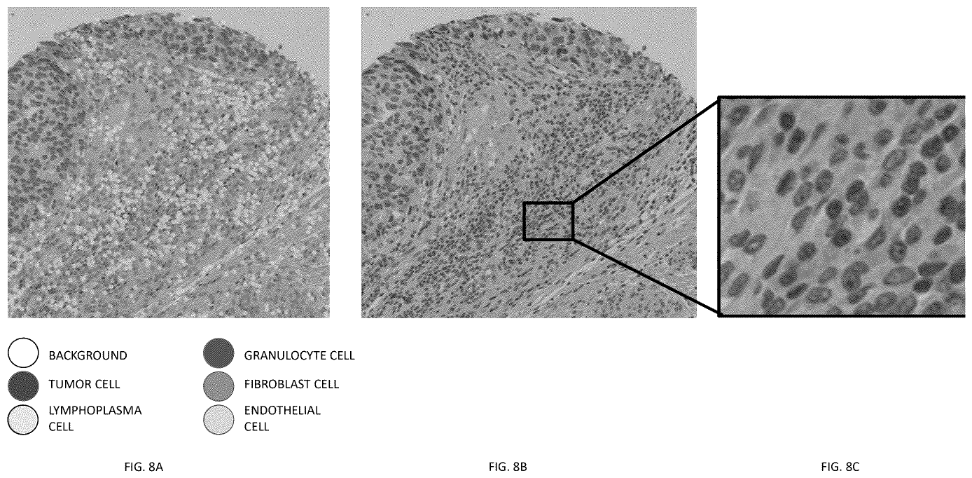

Reference is also made to FIG. 8A which is an image of an HE slide, labeled or annotated 113L by cell type classification module 113, according to some embodiments of the invention. As shown in the example of FIG. 8A, the labeling 113L is presented by color-coded cell types, where tumor cells are marked in red, Lymphoplasma cells are marked in yellow, Granulocyte cells are marked in blue, Fibroblast cells are marked in dark green, and Endothelial cells are marked in light green.

Reference is also made to FIGS. 8B and 8C which respectively depict an overview image, and a zoomed-in image of the HE slide of FIG. 8A (without the color-coded annotations 113L). In FIGS. 8B and 8C, nuclei of biological cells are segmented 122NS by segmentation module 122, according to some embodiments.

Reference is now made to FIGS. 9A-9C. FIG. 9A depicts three examples of HE slides that may be analyzed by embodiments of the invention to detect TLS regions.

FIG. 9B depicts three images, corresponding to the example images of FIG. 9A. As shown in FIG. 9B, candidate regions 110CR of interest were identified by embodiments of the invention, and are presented by red regions.

FIG. 9C depicts three lymphocyte density maps 114M that may be produced by density map generator 114 as elaborated herein, in respect to corresponding images of FIG. 9A, according to some embodiments of the invention.

Reference is now made back to FIG. 4, depicting an example of system 10. As shown in FIG. 4, system 10 may include a recommendation module 150, configured to produce a status notification data element 150SN, based on the classification 140CL of one or more candidate regions 110CR in one or more HE slides or images 20. Status notification data element 150SN may then be presented to a user on an output device (output 8 of FIG. 1) such as a screen (hence the term “notification”).

For example, recommendation module 150 may include or implement rule-based logic to determine a diagnosis of the subject based on a classification 140CL of one or more candidate regions 110CR (e.g., as TLS), the number of such identified candidate regions 110CR, the area of these candidate regions 110CR, and the like.

In another example, recommendation module 150 may include or implement a machine-learning based function to suggest treatment for the subject based on features (e.g., classifications 140CL, extent, area, and the like) of identified candidate regions 110CR.

In another example, recommendation module 150 may include or implement a machine-learning based function to predict a prognosis of the subject based on features (e.g., classifications 140CL, extent, area, and the like) of identified candidate regions 110CR. Specifically pertaining to prognostic value: The inventors have experimentally analyzed HE slides taken from Colorectal Cancer patients and found that median overall survival was significantly higher in patients with at least one predicted TLS (n=13) vs. patients with at least one predicted LA (n=13) detected on H&E (NR vs. 19 months, HR=0.21, 95% CI 0.06-0.78; p=0.01). In other words, embodiments of the invention have demonstrated the presence of TLS as a positive prognostic factor. Other such recommendations or notifications may be similarly implemented.

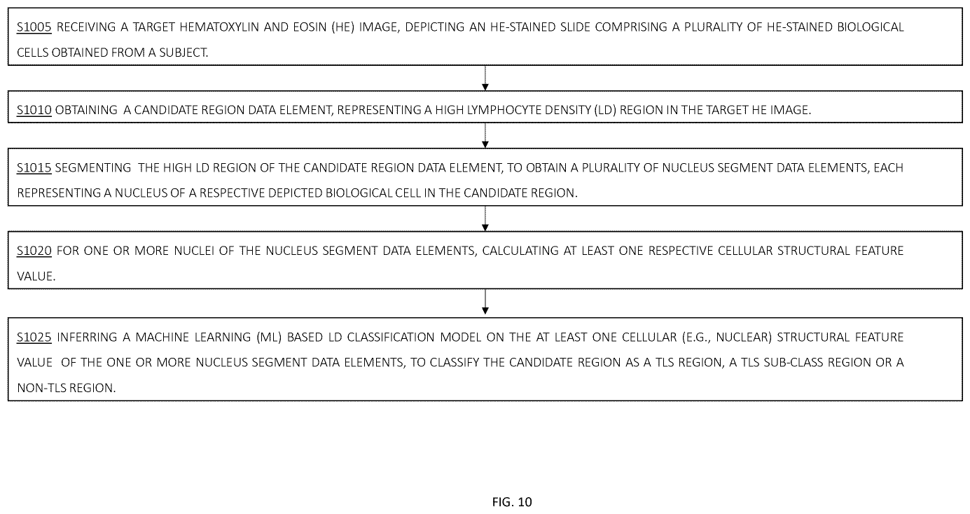

Reference is now made to FIG. 10 which is a schematic flow diagram depicting a method of identifying a TLS region in an HE slide obtained from a subject, by at least one processor (e.g., processor 2 of FIG. 1), according to some embodiments of the invention.

As shown in step S1005, the at least one processor 2 may receive a target Hematoxylin and Eosin (HE) image (image 20 of FIG. 4), depicting an HE-stained slide that includes a plurality of HE-stained biological cells obtained from a subject.

As shown in step S1010, the at least one processor 2 may obtain a candidate region data element 110CR, as elaborated herein, e.g., in relation to candidate region identifier 110 of FIG. 4. Candidate region data element 110CR may represent a high Lymphocyte Density (LD) region in the target HE image 20. As explained herein, the term “candidate region 110CR” may be used herein to refer to both candidate region data element 110CR, and the depicted region in image 20 which it represents.

As shown in step S1015, the at least one processor 2 may segment the high LD region of the candidate region data element, to obtain a plurality of cellular segment data elements (e.g., 112CS of FIG. 5). One or more (e.g., each) cellular segment data element 112CS may represent a respective depicted biological cell in the candidate region 110CR.

As shown in step S1020, the at least one processor 2 may calculate, for one or more biological cells of the cellular segment data elements 112CS, at least one respective cellular structural feature value (e.g., 124CF, 124SCF), as elaborated herein (e.g., in relation to cellular feature extraction module 124 of FIG. 4.

As shown in step S1025, the at least one processor 2 may infer an ML based LD classification model (e.g., 140 of FIG. 4) on the at least one cellular structural feature value 124CF/124SCF of the one or more cellular segment data elements 112CS. The at least one processor 2 may thereby classify the candidate region 110CR, e.g., as a TLS region, a TLS sub-class region or a non-TLS region.

As elaborated herein, embodiments of the invention may provide a practical application in the technological field of assistive diagnostics, to automatically classify pathology slides and/or regions thereof.

For example, embodiments of the invention may facilitate efficient screening of subjects to identify TLS, and distinguish or classify between TLS sub-classes. Embodiments of the invention may do so based on the commonly used HE staining procedure, instead of relying on more elaborate and expensive staining techniques, thereby reducing time, expenditure and expertise in the screening process.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A method of identifying a Tertiary Lymphoid Structure (TLS) by at least one processor, the method comprising:
receiving a target Hematoxylin and Eosin (HE) image, depicting an HE-stained slide comprising a plurality of HE-stained biological cells obtained from a subject;
obtaining a candidate region data element, representing a high Lymphocyte Density (LD) region in the target HE image;
segmenting the high LD region of the candidate region data element, to obtain a plurality of cellular segment data elements, each representing a respective depicted biological cell in the candidate region;
for one or more biological cells of the cellular segment data elements, calculating at least one respective cellular structural feature value; and
inferring a Machine Learning (ML) based LD classification model on the at least one cellular structural feature value of the one or more cellular segment data elements, to classify the candidate region as a TLS region, a TLS sub-class region or a non-TLS region.

2. The method of claim 1, further comprising segmenting the high LD region of the candidate region data element, to obtain a plurality of nucleus segment data elements, each representing a nucleus of a respective depicted biological cell in the candidate region, and wherein at least one cellular structural feature value represents a nucleus structure of one or more nuclei of the nucleus segment data elements.

3. The method of claim 1, wherein obtaining the candidate region data element comprises inferring an ML based region detection model on the target HE image, to obtain the candidate region data element,
wherein the region detection model is pretrained to detect regions of high LD in HE images, based on a plurality of training HE images.

4. The method of claim 3, wherein training the region detection model comprises:
receiving a plurality of training HE images, and a plurality of LD region annotations, corresponding to the plurality of training HE images, wherein at least one LD region annotation defines a region of high LD in a corresponding training HE image; and
using the plurality of LD region annotations as supervisory data, to train the region detection model to detect regions of high LD in the plurality of training HE images.

5. The method of claim 1 , wherein obtaining the candidate region data element comprises:
obtaining a plurality of cell type labels, wherein each cell type label defines a cell type of a respective biological cell, depicted in the target HE image;
producing a density map, representing density of lymphocyte cells based on the plurality of cell type labels; and
identifying a high LD region in the target HE image based on the density map.

6. The method of claim 5, wherein obtaining a cell type label comprises:
segmenting the target image to one or more cell segments, each comprising a cell depicted in the target image; and
inferring an ML based cell classification model on the one or more cell segments to obtain the cell type labels,
wherein the cell classification model is pretrained to classify cell segments in HE images, based on a plurality of annotated, training cell segments.

7. The method of claim 6, wherein training the cell classification model comprises:
receiving a plurality of training cell segments, each depicting a biological cell;
receiving a plurality of cell type annotations, each defining a cell type of a respective biological cell, depicted in the training HE image; and
using the cell type annotations as supervisory data for training the cell classification model to classify biological cells depicted in the training cell segments, according to cell types.

8. The method of claim 3, further comprising:
inferring the region detection model on a training HE image of the plurality of training HE images, to obtain a candidate region data element, representing a high LD region in the training HE image;
obtaining a plurality of cellular segment data elements, each representing a respective biological cell in the high LD region of the candidate region data element;
for one or more cells of the cellular segment data elements, calculating at least one respective cellular structural feature value;
receiving a TLS annotation data element, labelling the high LD region of the candidate region data element as representing either (i) a TLS region, (ii) a TLS sub-class region, or (iii) a non-TLS region; and
using the TLS annotation data element as supervisory data for training the LD classification model to classify the high LD region of the candidate region data element as a TLS region, a TLS sub-class region, or a non-TLS region, based on the cellular structural feature values.

9. The method of claim 1, wherein the cellular structure features are selected from a list consisting of a cell's size, a cell's roundness, a cell's eccentricity, a cell's convexity, a ratio between different areas of the cell, a cell's mean intensity, a cell's intensity variance, a cell's intensity range, a cell's color, a cell's color range, and any combination thereof.

10. The method of claim 1, wherein the cellular structure features represent structural features of cell-specific nuclei, selected from a list consisting of: nucleus convexity, nucleus eccentricity, nuclear area, nuclear solidity, nuclear color intensity variance, nuclear color intensity range, and a nuclear color intensity mean value.

11. The method of claim 1, wherein the cellular structure feature is a summary cellular structure feature, selected from a mean, a median and a standard deviation of a plurality of cell-specific cellular structure features, pertaining to a plurality of cells in one or more candidate regions.

12. The method of claim 1, further comprising:
calculating at least one morphology data element, representing a morphology of the candidate region; and
inferring the ML based LD classification model further on the at least one morphology data element, to classify the candidate region as a TLS region, a TLS sub-class region, or a non-TLS region.

13. The method of claim 12, wherein said morphology data element is selected from a list consisting of: an area of the candidate region, a cell count within the candidate region, a convexity of a polygon representing a circumference of the candidate region, a solidity of the polygon, and an eccentricity of the polygon.

14. The method of claim 1, further comprising producing a status notification, based on the classification of one or more candidate regions in one or more slides, wherein the status notification is selected from a list consisting of: a diagnosis of the subject, a prognosis of the subject, and a suggested treatment for the subject.

15. The method of claim 1, wherein calculating the cellular structural feature value comprises:
for at least one cellular segment data element, classifying the respective, depicted biological cell according to a cell type of a predefined set of cell types; and
selectively calculating the cellular structural feature value based on said classification.

16. A system for identifying a Tertiary Lymphoid Structure (TLS), the system comprising: a non-transitory memory device, wherein modules of instruction code are stored, and at least one processor associated with the memory device, and configured to execute the modules of instruction code, whereupon execution of said modules of instruction code, the at least one processor is configured to:
receive a target Hematoxylin and Eosin (HE) image, depicting an HE-stained slide comprising a plurality of HE-stained biological cells obtained from a subject;
obtain a candidate region data element, representing a high Lymphocyte Density (LD) region in the target HE image;
segment the high LD region of the candidate region data element, to obtain a plurality of cellular segment data elements, each representing a respective depicted biological cell in the candidate region;
for one or more biological cells of the cellular segment data elements, calculate at least one respective cellular structural feature value; and
infer a Machine Learning (ML) based LD classification model on the at least one cellular structural feature value of the one or more cellular segment data elements, to classify the candidate region as a TLS region, a TLS sub-class region or a non-TLS region.

17. The system of claim 16, further comprising segmenting the high LD region of the candidate region data element, to obtain a plurality of nucleus segment data elements, each representing a nucleus of a respective depicted biological cell in the candidate region, and wherein at least one cellular structural feature value represents a nucleus structure of one or more nuclei of the nucleus segment data elements.

18. The system of claim 16, wherein the at least one processor is configured to obtain the candidate region data element by inferring an ML based region detection model on the target HE image, to obtain the candidate region data element,
wherein the region detection model is pretrained to detect regions of high LD in HE images, based on a plurality of training HE images.

19. The system of claim 15, wherein the at least one processor is configured to train the region detection model by:
receiving a plurality of training HE images, and a plurality of LD region annotations, corresponding to the plurality of training HE images, wherein at least one LD region annotation defines a region of high LD in a corresponding training HE image; and
using the plurality of LD region annotations as supervisory data, to train the region detection model to detect regions of high LD in the plurality of training HE images.

20. The system of claim 16, wherein the at least one processor is configured to obtain the candidate region data element by:
obtaining a plurality of cell type labels, wherein each cell type label defines a cell type of a respective biological cell, depicted in the target HE image;
producing a density map, representing density of lymphocyte cells based on the plurality of cell type labels; and
identifying a high LD region in the target HE image based on the density map.

21-30. (canceled)
